(19) United States
(12) Patent Application Publication
(10) Pub. No.: US 2026/0283744 A1
(43) Pub. Date: Sep. 24, 2026

(54) MODULAR KIT FOR PROVIDING GUIDING DEVICES

(71) Applicant: EXOCAD GMBH, Darmstadt (DE)

(72) Inventor: Marco ANNUCCI, Rome (IT)

(21) Appl. No.: 19/568,220

(22) Filed: Mar. 16, 2026

(30) Foreign Application Priority Data
Mar. 18, 2025 (DE) DE202025101458.0

(51) Int. Cl. A61C 1/08; A61C 5/00; A61C 5/30; A61C 13/34

(52) U.S. Cl. CPC A61C 1/082; A61C 5/007; A61C 5/30; A61C 13/34

(57) ABSTRACT

Disclosed is a modular kit for providing guiding devices (100) for processing steps on oral tissue of a patient's jaw (150), which comprises, as a common basis for the guiding devices (100), a positioning splint (110) in the form of a patient-specific bite splint with a recess (112) as a common basis for the guiding devices (100), which is designed to expose an area of the patient's jaw (150) when the bite splint is placed on the patient's jaw (150). Furthermore, the modular kit comprises sets of at least one exchangeable patient-specific guiding insert (120) each. The guiding inserts (120) of the individual sets are positionable at predefined positions relative to the patient's jaw (150) by inserting them into the recess (112) of the bite splint. Each of the sets of guiding inserts (120) is assigned to one of the processing steps, and the guiding insert (120) of the corresponding set, when inserted, is configured to provide guidance for one or more instruments (130) during the processing step to which the corresponding set is assigned.

### CROSS REFERENCE TO RELATED APPLICATION

The present application claims priority to German Patent Application Serial No. 202025101458.0 filed Mar. 18, 2025, the entire contents of which are incorporated herein by reference.

### TECHNICAL FIELD

The invention relates to the field of dental technology, more specifically to a modular kit for providing guiding devices for processing steps on oral tissue of a patient's jaw.

### BACKGROUND

Dental processing of oral tissue in a patient's jaw poses a considerable challenge in terms of precision and care for the dentist performing the processing. In order to improve the quality of the results of such processing, guiding devices are sometimes provided to assist in guiding dental instruments during the processing.

### SUMMARY

One object of the invention is to provide a modular kit for the improved provision of guiding devices for processing steps on oral tissue of a patient's jaw. Furthermore, it is an object of the invention to provide a computer device and a computer program product for designing a corresponding modular kit. Finally, it is an object of the invention to provide a manufacturing system for manufacturing the corresponding modular kit.

The objects underlying the invention are solved by the features of the independent claims.

Disclosed is a modular kit for providing guiding devices for processing steps on oral tissue of a patient's jaw. The kit comprises, as a common basis for the guiding devices, a positioning splint in the form of a patient-specific bite splint with a recess which is designed to expose an area of the patient's jaw when the bite splint is placed on the patient's jaw.

The kit also comprises sets of at least one exchangeable patient-specific guiding insert each. The guiding inserts of the individual sets are positionable at predefined positions relative to the patient's jaw by inserting them into the recess of the bite splint. Each of the sets of guiding inserts is assigned to one of the processing steps, and the guiding insert of the corresponding set is configured, when inserted, to provide guidance for one or more instruments in the course of the processing step to which the corresponding set is assigned.

The bite splint is reconfigurable by exchanging the one or more guiding inserts of one of the sets assigned to one processing step, which are arranged in the recess, with the one or more guiding inserts of another one of the sets, which is assigned to another one of the processing steps, from a configuration as a guiding device for the one processing step into another guiding device for the other processing step.

Examples could have the advantage that a positioning splint is provided in the form of a patient-specific bite splint, which is configured to fit precisely onto the patient's jaw. The recess in the positioning splint or bite splint exposes, for example, an area of the patient's jaw that comprises oral tissue to be processed in the course of a plurality of processing steps.

Furthermore, a plurality of sets of guiding inserts is provided for the positioning splint. The individual guiding inserts may be inserted into the recess of the bite splint, for example. By inserting them into the recess, the guiding inserts are positioned at predefined positions relative to the patient's jaw and may be connected to the bite splint at these predefined positions in a non-destructive, detachable manner. As a result of the non-destructive detachable connection, one set of guiding inserts may be exchanged for another set of guiding inserts, for example. By exchanging the guiding inserts, the bite splint may be converted from a configuration as a first guiding device to a configuration as a second guiding device. Thus, using a modular kit, a plurality of guiding devices may be provided for performing a plurality of processing steps. In particular, by providing more than two sets of guiding inserts, more than two guiding devices may be provided for performing more than two processing steps. For example, the modular kit may comprise two, three, four, five, six, seven, eight, nine, ten, or more sets of guiding inserts for providing two, three, four, five, six, seven, eight, nine, ten, or more guiding devices for performing two, three, four, five, six, seven, eight, nine, ten, or more processing steps.

The oral tissue may, for example, comprise a jawbone, a gingiva, and/or one or more teeth. The processing steps may, for example, comprise one or more processing steps for processing the jawbone, one or more processing steps for processing the gingiva, and/or one or more processing steps for processing the one or more teeth.

The processing steps may, for example, comprise one or more preparation steps for preparing one or more sections of the jawbone to be prepared within the recess. The processing steps may, for example, comprise one or more inspection steps for inspecting preparation results, such as preparation depths and/or prepared shapes of one or more prepared sections of the jawbone within the recess.

The processing steps may, for example, comprise one or more preparation steps for preparing one or more sections of one or more teeth to be prepared within the recess. The processing steps may, for example, comprise one or more inspection steps for inspecting preparation results, such as preparation depths and/or prepared shapes of one or more prepared teeth within the recess.

The processing steps may, for example, comprise one or more reduction steps for reducing or cutting one or more sections of the gingiva to be reduced within the recess. The processing steps may, for example, comprise one or more inspection steps for inspecting reduction results of one or more sections of the gingiva to be reduced within the recess.

The processing steps may, for example, comprise one or more build-up steps for building up one or more sections to be built up on one or more teeth within the recess. The one or more build-up steps comprise, for example, applying build-up material to the corresponding teeth. The processing steps may, for example, comprise one or more inspection steps for inspecting build-up results, such as build-up thicknesses and/or built-up shapes of one or more built-up teeth within the recess.

The processing steps may, for example, comprise one or more trial restoration steps for applying trial restoration material for one or more trial restoration parts to one or more teeth within the recess. The processing steps may, for example, comprise one or more inspection steps for inspecting trial restoration results, such as trial restoration thickness and/or trial restoration shapes of one or more trial restoration parts on one or more teeth within the recess.

The processing steps may, for example, comprise one or more trial restoration steps for applying or positioning one or more trial restoration parts on one or more teeth within the recess. The processing steps may, for example, comprise one or more inspection steps for inspecting one or more positions of one or more trial restoration parts on one or more teeth within the recess.

The processing steps may, for example, comprise one or more drilling steps for drilling one or more drill holes in oral tissue of the patient's jaw within the recess, for example in one or more teeth and/or in a bone section of the patient's jaw within the recess. The processing steps may, for example, comprise one or more inspection steps for inspecting drilling results, such as positions, diameters, angles, and/or depths of drill holes in one or more teeth and/or in a bone section of the patient's jaw within the recess.

The processing steps may, for example, comprise one or more restoration steps for applying or positioning one or more restoration parts on one or more prepared teeth within the recess. The processing steps may, for example, comprise one or more inspection steps for inspecting one or more positions of one or more restoration parts on one or more prepared teeth within the recess. The corresponding restoration parts comprise, for example, veneers, crowns, inlays, onlays, and/or overlays.

The processing steps may, for example, comprise one or more positioning steps for applying or positioning one or more orthodontic appliances on one or more teeth within the recess. The processing steps may, for example, comprise one or more inspection steps for inspecting one or more positions of one or more orthodontic appliances on one or more teeth within the recess. The orthodontic appliances serve, for example, to correct the position of one or more teeth within the recess. The corresponding orthodontic appliances comprise, for example, brackets for fixed braces.

The guiding inserts may be patient-specific guiding inserts, each of which is configured to achieve a patient-specific processing result to be achieved for a processing step on the oral tissue of the patient's jaw to be processed, or to check whether the patient-specific processing result to be achieved has been achieved. For this purpose, the patient-specific guiding inserts may be configured to guide an instrument for achieving the patient-specific processing result to be achieved and/or to guide an instrument for checking whether the patient-specific processing result to be achieved has been achieved.

Examples could have the advantage of achieving individual adaptation of the guiding devices provided by the modular kit to the patient's jaw, since the patient-specific bite splint and the exchangeable, precisely positionable guiding inserts may enable a precise fitting orientation to the patient's jaw. In particular, this individual adaptation may be achieved for a plurality of guiding devices.

Examples could have the advantage of enabling a flexible change from one guiding device for one processing step to another guiding device for another processing step, since the modular exchange of guiding inserts allows different processing steps to be efficiently mapped. Thus, a modular kit may enable a plurality of guiding splints for performing a plurality of processing steps.

Examples could have the advantage that the same positioning splint may be used as a common basis for all guiding devices and thus for all processing steps. This allows, for example, the positioning splint to be positioned once on the patient's jaw. The positioning splint may then be converted to the different guiding devices for the different processing steps by exchanging the guiding inserts. In this case, the position of the positioning splint on the patient's jaw may be maintained, for example. Alternatively, the positioning splint may be removed from the patient's jaw to exchange the guiding inserts. The use of the same positioning splint allows for easy repositioning in each case.

In one example, the bite splint comprises at least a first connecting element at an edge of the recess. The guiding inserts of the sets each comprise at least a second connecting element at an edge. The first and second connecting elements are configured to form non-destructively detachable first connections between at least some of the guiding inserts of the sets and the bite splint in order to hold the corresponding guiding inserts in position when the same are each arranged in the recess of the bite splint.

Examples could have the advantage that the first connecting element at a defined edge area of the recess supports precise fixation of a guiding insert with the second connecting element. This allows a stable connection between the bite splint and the guiding inserts to be achieved.

Examples could have the advantage that the exchanging of the guiding inserts is facilitated, since the clearly defined connecting elements allow easy assembling and disassembling of the guiding inserts in the positioning splint.

In one example, at least some of the second connecting elements are further configured to form non-destructively detachable second connections between guiding inserts of the sets arranged in adjacent positions within the recess of the bite splint in order to hold the corresponding guiding inserts in the corresponding positions.

Examples could have the advantage that additional mechanical stability may be achieved by means of additional connections between adjacent guiding inserts.

Examples could have the advantage that the positional stability of the guiding inserts as a whole and relative to each other may be increased.

In one example, the first connecting element is arranged in an area of the edge of the recess arranged on an oral side of the recess.

Examples could have the advantage that arranging the first connecting element on an oral side of the recess may ensure that access to the recess and thus to the oral tissue exposed by the recess is not blocked from the occlusal and, in particular, vestibular direction. This allows the guiding inserts to encompass recesses, for example, which enable instruments to be guided in the occlusal and/or vestibular direction.

Examples could have the advantage that anatomically correct alignment of the guiding inserts may be achieved. The functional integration of the guiding inserts may be improved, since the side-specific placement of the first connecting elements provides clear spatial orientation.

In one example, at least some of the second connecting elements are each arranged in an area of the edges of the guiding inserts located on an oral side of the guiding inserts.

Examples could have the advantage that an arrangement of second connecting elements on an oral side of the guiding inserts may ensure that access to the recess and thus to the oral tissue exposed by the recess is not blocked from the occlusal and, in particular, vestibular direction. This allows the guiding inserts to encompass, for example, recesses that guide instruments in the occlusal and/or vestibular direction.

Examples could have the advantage that anatomically correct alignment of the guiding inserts may be achieved. The functional integration of the guiding inserts may be improved, since the side-specific placement of the second connecting elements provides clear spatial orientation.

In one example, the first connecting element is arranged in an area of the edge of the recess located on a vestibular side of the recess.

Examples could have the advantage that anatomically correct alignment of the guiding inserts may be achieved. The functional integration of the guiding inserts may be improved, since the side-specific placement of the first connecting elements provides clear spatial orientation.

Examples could have the advantage that a vestibular arrangement of the first connecting element may facilitate exchanging of the guiding inserts while the positioning splint is positioned on the patient's jaw. A vestibular arrangement of the first connecting element may increase external accessibility to a non-destructively detachable first connection comprising the first connecting element while the positioning splint is positioned on the patient's jaw.

In one example, at least some of the second connecting elements are each arranged in an area of the edges of the guiding inserts arranged on a vestibular side of the guiding inserts.

Examples could have the advantage that anatomically correct alignment of the guiding inserts may be achieved. The functional integration of the guiding inserts may be improved, since the side-specific placement of the second connecting elements provides clear spatial orientation.

Examples could have the advantage that a vestibular arrangement of the second connecting elements may facilitate exchanging of the guiding inserts while the positioning splint is positioned on the patient's jaw. A vestibular arrangement of the second connecting element may increase external accessibility to a non-destructively detachable first connection comprising the second connecting element while the positioning splint is positioned on the patient's jaw.

In one example, at least a pair of first connecting elements is arranged in areas of the edge of the recess located on opposite distal sides of the recess.

Examples could have the advantage that a higher structural symmetry and thus a balanced load distribution may be achieved. The pair of first connecting elements on opposite distal sides of the recess may enable a balanced load distribution on the two distal sides of the recess.

Examples could have the advantage of increasing the overall stability of the guiding devices, since the symmetrical arrangement of the connecting elements may contribute to an improved spatial fixation of the guiding inserts in the recess.

In one example, at least some of the second connecting elements are arranged in pairs in areas of the edges of the guiding inserts located on opposite distal sides of the corresponding guiding inserts.

Examples could have the advantage that a higher structural symmetry and thus a balanced load distribution may be achieved. The paired arrangement of second connecting elements on opposite distal sides of the guiding inserts enables a balanced load distribution on the two distal sides of the guiding inserts.

Examples could have the advantage that the overall stability of the guiding devices is increased in each case, since the symmetrical arrangement of the connecting elements may contribute to an improved spatial fixation of the guiding inserts. The paired arrangement of second connecting elements on opposite distal sides of the guiding inserts may contribute to an improved spatial fixation of the guiding inserts to each other and/or to the positioning splint.

In one example, the first and second connecting elements and/or second connecting elements form pairs of mutually assigned connecting elements. The pairs each comprise a projection and a receptacle designed to be complementary to the projection. The pairs are each configured to form one of the non-destructively detachable first or second connections.

Examples could have the advantage that, using corresponding pairs of complementary connecting elements, a simple but at the same time secure and detachable connection may be established. The combination of projection and complementary receptacle may enable precise and gentle coupling of the connecting elements and thus of the guiding inserts to the positioning splint and/or of the guiding inserts to each other.

Examples could have the advantage that the guiding inserts may be exchanged in the positioning splint without structural impairment, as the corresponding non-destructive detachable connections support a simple and reversible assembling process for the guiding inserts.

In one example, the receptacles are designed as grooves and the projections as tongues. The pairs consisting of a projection and a receptacle designed to complement the projection are provided, for example, as pairs of complementary tongues and grooves. A tongue and groove design may, for example, have the advantage of providing a type of rail system in which the components connected by the corresponding connecting elements, in particular the guiding inserts, may be pushed into each other along a longitudinal direction of the tongue and groove. This may have the advantage that a single guiding insert can be easily and selectively pulled out again.

The corresponding first and/or second connecting elements each have, for example, an elongated shape in a longitudinal direction. The longitudinal direction of the corresponding connecting elements and thus the insertion direction of the guiding inserts may be, for example, a direction perpendicular to an occlusal surface and towards the patient's jaw. The longitudinal direction of the corresponding connecting elements and thus the insertion direction of the guiding inserts may, for example, be an oral or vestibular direction.

For example, the first connecting elements and at least some of the second connecting elements form pairs of mutually assigned connecting elements, each of the pairs comprising a first connecting element and a complementarily designed second connecting element. For example, the first connecting elements are each designed as projections and the corresponding second connecting elements are each designed as complementary receptacles. For example, the corresponding second connecting elements are each designed as projections and the first connecting elements are each designed as complementary receptacles.

For example, some of the first connecting elements are designed as projections and some of the first connecting elements are designed as receptacles, while some of the corresponding complementary second connecting elements are designed as receptacles and some of the corresponding complementary second connecting elements are designed as projections. In this case, for example, a projection of the first connecting elements is in each case assigned to a receptacle of the second connecting elements and a receptacle of the first connecting elements is assigned to a projection of the second connecting elements.

For example, at least some of the second connecting elements form pairs of mutually assigned second complementary connecting elements. Each of the corresponding pairs comprises, for example, a second connecting element configured as a projection and a second connecting element designed as a complementary receptacle. For example, the pairs of second connecting elements are each second connecting elements of guiding inserts to be arranged in adjacent positions within the recess of the bite splint.

For example, the projections and complementary receptacles may be designed similarly to connecting elements of puzzle pieces, i.e., like a puzzle. For example, the projections and complementary receptacles may be designed to form non-destructive detachable pin and socket connections. In this case, the projections are configured, for example, as bulges or pins, in particular as flat pin-shaped bulges, and the receptacles as complementary indentations or sockets for receiving the corresponding bulges or pins. Each of the pin and socket connections consists, for example, of a pin and an assigned complementary socket. For example, the shape of the pins and complementary sockets may vary slightly from connection to connection in order to ensure a unique fit for each connection. This prevents, for example, guiding inserts that are not intended to be arranged next to each other from being accidentally combined or arranged next to each other. Furthermore, it may be prevented, for example, that guiding inserts are accidentally arranged in incorrect positions within the recess. The pins, for example, each protrude beyond the corresponding component of the modular kit, i.e., the edge of the recess of the bite splint and/or the edges of guiding inserts. For example, the pins each have a rounded and/or slightly oval shape. For example, the pins are drop-shaped. For example, the pins are each designed to fit exactly into a corresponding socket.

The receptacles are configured, for example, as sockets complementary to the pins, i.e., recesses in the edge or rim of the corresponding components of the modular kit, i.e., the recess of the bite splint and/or the guiding inserts. The shape of the sockets in each case corresponds exactly to the shape of an assigned matching pin, so that they may fit together and form a pin and socket connection. For example, the various pin and socket connections may each comprise differently shaped pins and sockets, for example larger/smaller, narrower/wider and/or asymmetrically shaped pins and sockets, in order to prevent accidental misconnections between components of the modular kit that do not belong together. The accuracy of fit may, for example, prevent incorrect components of the modular kit from being assembled.

Due to their accuracy of fit, pin and socket connections may, for example, prevent incorrect guiding inserts from being accidentally connected to each other and/or guiding inserts from being positioned in the wrong position within the recess of the bite splint. The size and shape of the pins and sockets may be designed in such a way that a clear connection is only possible between matching pins and sockets.

Due to their stability, pin and socket connections may ensure that the positioning splint with inserted guiding elements provides a stable guiding device, which enables stable guidance of instruments during one of the processing steps. Slight friction between the connecting elements and/or between the edges of the interconnected components of the modular kit, i.e., the bite splint and/or the guiding inserts, may ensure that the corresponding components do not easily detach from each other.

For example, the edge of the recess may have a chamfer or be chamfered so that the edge of the recess tapers from an outer surface of the bite splint toward the recess or toward the inner surface of the bite splint. The outer surface is a surface of the bite splint that faces away from the patient's jaw when the bite splint is positioned on the patient's jaw as intended. The inner surface is a surface of the bite splint that rests on the patient's jaw when the bite splint is positioned on the patient's jaw as intended. The edges of the guiding inserts that are in contact with the edge of the bite splint have complementary chamfers, for example. For example, corresponding chamfers ensure that the guiding inserts cannot be pressed into the recess of the bite splint when the same are arranged in the recess.

In one example, the recess of the bite splint is designed to expose a plurality of teeth and/or a portion of a gingiva and/or a portion of a jawbone of the patient's jaw.

Examples could have the advantage that the recess, with its design for exposing a plurality of teeth and/or parts of the gingiva and/or parts of the jawbone, may ensure accessibility to the corresponding plurality of teeth and/or the corresponding parts of the gingiva and/or the corresponding parts of the jawbone. Guiding inserts arranged in the recess may thus provide guidance for one or more instruments in the course of processing the corresponding plurality of teeth and/or the corresponding parts of the gingiva and/or the corresponding parts of the jawbone.

In one example, the sets of guiding inserts comprise guiding inserts of different types for different processing steps of different types.

Examples could have the advantage that a versatile application of the modular kit or the different guiding devices implementable by means of the different guiding inserts may be achieved. The integration of different types of guiding inserts for specific processing steps may enable individual adaptation of the guiding devices to different clinical requirements.

Examples could have the advantage that the resulting different guiding devices may be used for different diagnostic and therapeutic purposes in the course of the processing steps. The individual processing steps may be optimized by using specialized guiding inserts for each of them.

For example, the sets of guiding inserts comprise one or more sets for processing the jawbone, one or more sets for processing the gingiva, and/or one or more sets for processing one or more teeth.

In one example, the sets of guiding inserts comprise a set with at least one guiding insert for identifying one or more sections of the jawbone to be prepared within the recess. The guiding inserts may, for example, comprise one or more recesses whose contours define contours to be prepared of the sections to be prepared. In one example, the sets of guiding inserts comprise a plurality of corresponding sets for a plurality of corresponding identification steps.

In one example, the sets of guiding inserts comprise a set with at least one guiding insert for inspecting preparation depths of one or more prepared sections of the jawbone within the recess. The guiding inserts may, for example, comprise one or more recesses for receiving one or more instruments for measuring the preparation depths. In one example, the sets of guiding inserts comprise a plurality of corresponding sets for a plurality of corresponding inspection steps.

In one example, the sets of guiding inserts comprise a set with at least one guiding insert for inspecting shapes of one or more prepared sections of the jawbone within the recess. The guiding inserts may, for example, comprise one or more recesses whose contours define the shapes to be prepared or contours of the shapes to be prepared as reference contours. In one example, the sets of guiding inserts comprise a plurality of corresponding sets for a plurality of corresponding inspection steps.

In one example, the sets of guiding inserts comprise a set with at least one guiding insert for identifying one or more sections of one or more teeth to be prepared within the recess. The guiding inserts may, for example, comprise one or more recesses whose contours specify contours to be prepared of the sections to be prepared. In one example, the sets of guiding inserts comprise a plurality of corresponding sets for a plurality of corresponding identification steps.

In one example, the sets of guiding inserts comprise a set with at least one guiding insert for inspecting preparation depths of one or more prepared teeth within the recess. The guiding inserts may, for example, comprise one or more recesses for receiving one or more instruments for measuring the preparation depths. In one example, the sets of guiding inserts comprise a plurality of corresponding sets for a plurality of corresponding inspection steps.

In one example, the sets of guiding inserts comprise a set with at least one guiding insert for inspecting shapes of one or more prepared teeth within the recess. The guiding inserts may, for example, comprise one or more recesses whose contours define the shapes to be prepared or contours of the shapes to be prepared as reference contours. In one example, the sets of guiding inserts comprise a plurality of corresponding sets for a plurality of corresponding inspection steps.

In one example, the sets of guiding inserts comprise a set with at least one guiding insert for identifying one or more sections of the gingiva to be cut within the recess. The guiding inserts may, for example, comprise one or more recesses whose contours specify contours for cutting the sections of the gingiva to be cut. Furthermore, the recesses may, for example, also specify angles for cutting the gingiva sections. In one example, the sets of guiding inserts comprise a plurality of corresponding sets for a plurality of corresponding cutting steps.

In one example, the sets of guiding inserts comprise a set with at least one guiding insert for inspecting shapes of one or more cut sections of the gingiva within the recess. The guiding inserts may, for example, comprise one or more recesses whose contours define the shapes to be cut of the cut sections of the gingiva or their contours as reference contours. In one example, the sets of guiding inserts comprise a plurality of corresponding sets for a plurality of corresponding inspection steps.

In one example, the sets of guiding inserts comprise a set with at least one guiding insert for identifying one or more sections to be built up on one or more teeth within the recess. The guiding inserts may, for example, comprise one or more recesses whose contours specify contours to be achieved for the one or more sections to be built up. In one example, the sets of guiding inserts comprise a plurality of corresponding sets for a plurality of corresponding identification steps.

In one example, the sets of guiding inserts comprise a set with at least one guiding insert for inspecting build-up thicknesses of one or more built-up teeth within the recess. The guiding inserts may, for example, comprise one or more recesses for receiving one or more instruments for measuring the build-up thickness. In one example, the sets of guiding inserts comprise a plurality of corresponding sets for a plurality of corresponding inspection steps.

In one example, the sets of guiding inserts comprise a set with at least one guiding insert for inspecting shapes of one or more built-up teeth within the recess. The guiding inserts may, for example, comprise one or more recesses whose contours define the shapes to be built up or the contours of the shapes to be built up as reference contours. In one example, the sets of guiding inserts comprise a plurality of corresponding sets for a plurality of corresponding inspection steps.

In one example, the sets of guiding inserts comprise a set with at least one guiding insert for applying trial restoration material for one or more trial restoration parts to one or more teeth within the recess. The guiding inserts may, for example, comprise one or more recesses whose contours specify the contours of the one or more teeth to be achieved by applying the trial restoration material. In one example, the sets of guiding inserts comprise a plurality of corresponding sets for a plurality of corresponding build-up steps.

In one example, the sets of guiding inserts comprise a set with at least one guiding insert for inspecting trial restoration thicknesses of trial restoration material applied to one or more teeth within the recess. The guiding inserts may, for example, comprise one or more recesses for receiving one or more instruments for measuring the trial restoration thicknesses. In one example, the sets of guiding inserts comprise a plurality of corresponding sets for a plurality of corresponding inspection steps.

In one example, the sets of guiding inserts comprise a set with at least one guiding insert for inspecting shapes of trial restoration material applied to one or more teeth within the recess. The guiding inserts may, for example, comprise one or more recesses whose contours define the shapes or contours of the one or more teeth to be achieved by applying the trial restoration material as reference contours. In one example, the sets of guiding inserts comprise a plurality of corresponding sets for a plurality of corresponding inspection steps.

In one example, the sets of guiding inserts comprise a set with at least one guiding insert for positioning one or more trial restoration parts on one or more teeth within the recess. The guiding inserts may, for example, comprise one or more recesses whose contours specify positions for the one or more trial restoration parts on the one or more teeth. In one example, the sets of guiding inserts comprise a plurality of corresponding sets for a plurality of corresponding positioning steps.

In one example, the sets of guiding inserts comprise a set with at least one guiding insert for inspecting positions of one or more trial restoration parts on one or more teeth within the recess. The guiding inserts may, for example, comprise one or more recesses whose contours define reference contours for the one or more trial restoration parts, which the trial restoration parts should have when the same are correctly positioned on the one or more teeth. In one example, the sets of guiding inserts comprise a plurality of corresponding sets for a plurality of corresponding inspection steps.

In one example, the sets of guiding inserts comprise a set with at least one guiding insert for positioning one or more drills relative to the patient's jaw within the recess. The guiding inserts may, for example, comprise one or more recesses for receiving and guiding the one or more drills relative to the patient's jaw. In one example, the sets of guiding inserts comprise a plurality of corresponding sets for a plurality of corresponding positioning steps.

In one example, the sets of guiding inserts comprise a set with at least one guiding insert for inspecting drilling results of drill holes drilled into the patient's jaw by means of one or more drills. The guiding inserts may, for example, comprise one or more recesses for receiving one or more instruments for inspecting the drilling results. Inspecting the drilling results may, for example, comprise inspecting positions, diameters, angles, and/or depths of the drill holes. In one example, the sets of guiding inserts include a plurality of corresponding sets for a plurality of corresponding inspection steps.

In one example, the sets of guiding inserts comprise a set with at least one guiding insert for positioning one or more dental restoration parts on one or more prepared teeth within the recess. The guiding inserts may, for example, comprise one or more recesses whose contours specify positions for the one or more restoration parts on the one or more prepared teeth. In one example, the sets of guiding inserts comprise a plurality of corresponding sets for a plurality of corresponding positioning steps.

In one example, the sets of guiding inserts comprise a set with at least one guiding insert for inspecting positions of one or more restoration parts on one or more prepared teeth within the recess. The guiding inserts may, for example, comprise one or more recesses whose contours define reference contours for the one or more restoration parts, which the restoration parts should have when the same are correctly positioned on the one or more prepared teeth. In one example, the sets of guiding inserts comprise a plurality of corresponding sets for a plurality of corresponding inspection steps.

In one example, the sets of guiding inserts comprise a set with at least one guiding insert for positioning one or more orthodontic appliances on one or more teeth within the recess. The guiding inserts may, for example, comprise one or more recesses whose contours specify positions for the one or more orthodontic appliances on one or more teeth. In one example, the sets of guiding inserts comprise a plurality of corresponding sets for a plurality of corresponding positioning steps.

In one example, the sets of guiding inserts comprise a set with at least one guiding insert for inspecting positions of one or more orthodontic appliances on one or more teeth within the recess. The guiding inserts may, for example, comprise one or more recesses whose contours define positions for the one or more orthodontic appliances that the orthodontic appliances should have when correctly positioned on the one or more teeth. In one example, the sets of guiding inserts comprise a plurality of corresponding sets for a plurality of corresponding inspection steps.

Further disclosed is a computer device for designing a modular kit according to one of the preceding examples for providing multiple guiding devices for multiple processing steps on oral tissue of a patient's jaw. The computer device comprises a processor unit and a memory unit with stored machine-readable program instructions. An execution of the program instructions by the processor unit causes the processor unit to control the computer device to receive a three-dimensional digital jaw model of the patient's jaw. The three-dimensional digital jaw model comprises, for example, the oral tissue of the patient's jaw to be processed in the course of the multiple processing steps. Three-dimensional digital processing models of the patient's jaw are generated, each defining a target state for the multiple processing steps on the oral tissue of the patient's jaw. A three-dimensional digital positioning splint model of the positioning splint of the kit is generated using the three-dimensional digital jaw model as a digital template for manufacturing the positioning splint of the kit. Furthermore, sets of at least one three-dimensional digital guiding insert model each for the sets of exchangeable patient-specific guiding inserts of the kit are generated as digital templates for manufacturing the sets of guiding inserts of the kit.

Examples could have the advantage that precise, individual planning of the modular kit for a patient may be realized. In particular, individual digital templates for manufacturing the positioning splint and the sets of guiding inserts may be generated based on a three-dimensional digital jaw model of the patient's jaw. This enables, for example, a precise fit of the guiding devices implemented using the modular kit on the patient's jaw, as well as precise guidance of instruments through the corresponding guiding devices, depending on the individual conditions of the oral tissue of the patient's jaw to be processed.

Examples could have the advantage that manufacturing preparation may be accelerated by digital design of the modular kit. In addition, digital generation of a positioning splint model and guiding insert models as digital templates for manufacturing the positioning splint and sets of guiding inserts may enable efficient transfer to and/or control of manufacturing devices.

Examples could have the advantage of reducing development costs, as digital simulations and planning may minimize potential sources of error and optimize the manufacturing process.

For example, the program instructions are configured to control the computer device to construct a modular kit according to any of the examples of a modular kit described herein.

In one example, an execution of the program instructions by the processor unit further causes the processor unit to control one or more manufacturing devices to manufacture the positioning splint of the kit and the sets of exchangeable patient-specific guiding inserts of the kit as physical copies of the corresponding digital templates.

Examples may have the advantage of achieving seamless integration between digital planning and physical manufacturing of the modular kit. For example, automated control of the manufacturing devices by the computer device may ensure a consistent manufacturing process.

Examples could have the advantage of ensuring high quality of the manufactured components of the modular kit. In particular, high dimensional accuracy may be supported by the exact conversion of the digital templates into physical copies.

Examples could have the advantage that the manufacturing process may be made more flexible. For example, rapid adaptation and reproduction of components of the modular kit using digital template creation may support optimized manufacturing dynamics.

For example, the manufacturing of the components of the modular kit, i.e., the positioning splint of the kit and the sets of exchangeable patient-specific guiding inserts, comprises machining one or more blanks and/or 3D printing the corresponding components. The components may be manufactured as positives by means of machining and/or 3D printing. Alternatively, negatives of the components to be manufactured may be manufactured by means of machining and/or 3D printing. These negatives may be used as casting molds for manufacturing the corresponding components by means of a casting technique in which a casting material is inserted into the casting molds and cured.

The one or more manufacturing devices comprise, for example, one or more devices for machining, such as milling devices. A device for machining may be configured to remove material from a blank using one or more machining tools in order to manufacture a physical copy of a digital template. The one or more manufacturing devices include, for example, one or more 3D printing devices. A 3D printing device may, for example, be configured to print a physical copy of a digital template layer by layer.

Examples enable computer-aided design (CAD) of the modular kit for creating, modifying, analyzing, and/or optimizing three-dimensional digital models of the components of the modular kit. The three-dimensional digital models may define geometric shapes of the components, for example, surfaces of these components. For example, the surfaces of the components may be defined as a polygon mesh or point cloud. A polygon mesh comprises a collection of nodes, edges, and faces that define the shape of a surface of a polyhedral object. Furthermore, computer-aided manufacturing (CAM) may be used to manufacture physical copies of the three-dimensional digital models defined using CAD. In the course of computer-aided manufacturing, manufacturing devices, for example, devices for machining and/or 3D printing devices, are controlled for the manufacturing of physical copies defined by the three-dimensional digital models used as templates.

For example, the program instructions for controlling the manufacturing devices are configured to manufacture a modular kit according to any of the examples of a modular kit described herein.

Further disclosed is a manufacturing system for manufacturing a modular kit according to one of the examples of a modular kit described herein for providing multiple guiding devices for multiple processing steps on oral tissue of a patient's jaw. The manufacturing system comprises a computer device according to one of the examples of a computer device described herein and one or more manufacturing devices for manufacturing the positioning splint of the kit and the sets of exchangeable patient-specific guiding inserts of the kit.

Examples may have the advantage of enabling computer-aided manufacturing of the modular kit.

Examples could have the advantage of achieving seamless integration between digital planning and physical manufacturing of the modular kit. Automated control of the manufacturing devices by the computer device may, for example, ensure a consistent manufacturing process.

Examples could have the advantage of ensuring high quality of the manufactured components of the modular kit. In particular, high dimensional accuracy may be supported by the exact conversion of the digital templates into physical copies.

For example, the manufacturing system is configured for manufacturing a modular kit according to any of the examples of a modular kit described herein.

Further disclosed is a computer program product for designing a modular kit according to one of the examples of a modular kit described herein for providing multiple guiding devices for multiple processing steps on oral tissue of a patient's jaw. The computer program product comprises a computer-readable storage medium embodying machine-readable program instructions.

An execution of the program instructions by a processor unit of a computer device causes the processor unit to control the computer device to receive a three-dimensional digital jaw model of the patient's jaw. The three-dimensional digital jaw model comprises, for example, the oral tissue of the patient's jaw to be processed in the course of the multiple processing steps. Three-dimensional digital processing models of the patient's jaw are generated, each defining a target state for the multiple processing steps on the oral tissue of the patient's jaw. A three-dimensional digital positioning splint model of the positioning splint of the kit is generated using the three-dimensional digital jaw model as a digital template for manufacturing the positioning splint of the kit. Furthermore, sets of at least one three-dimensional digital guiding insert model each for the sets of exchangeable patient-specific guiding inserts of the kit are generated as digital templates for manufacturing the sets of guiding inserts of the kit.

Examples could have the advantage that manufacturing preparation may be accelerated by digital design of the modular kit. In addition, digital generation of a positioning splint model and guiding insert models as digital templates for manufacturing the positioning splint and the sets of guiding inserts may enable efficient transfer to and/or control of manufacturing devices.

Examples may have the advantage of reducing development costs, as digital simulations and planning may minimize potential sources of error and achieve an optimized manufacturing process.

For example, the program instructions are configured to control the computer device to construct a modular kit according to any of the examples of a modular kit described herein.

In one example, an execution of the program instructions by the processor unit further causes the processor unit to control one or more manufacturing devices to manufacture the positioning splint of the kit and the sets of exchangeable patient-specific guiding inserts of the kit as physical copies of the corresponding digital templates.

Examples could have the advantage of achieving seamless integration between digital planning and physical manufacturing of the modular kit. Automated control of the manufacturing devices equipment by the computer device may, for example, ensure a consistent manufacturing process.

Examples could have the advantage of ensuring high quality of the manufactured components of the modular kit. In particular, high dimensional accuracy may be supported by the exact conversion of digital templates into physical copies.

Examples could have the advantage that the manufacturing process may be made more flexible. For example, rapid adaptation and reproduction of components of the modular kit using digital template creation may support optimized manufacturing dynamics.

For example, the program instructions are configured for controlling the manufacturing devices for manufacturing a modular kit according to any of the examples of a modular kit described herein.

It is understood that one or more of the aforementioned embodiments may be combined with each other as long as the embodiments are not mutually exclusive.

### BRIEF DESCRIPTION OF THE DRAWINGS

In the following, examples are explained in more detail with reference to the drawings. They show:

FIG. 1 an exemplary guiding device of an exemplary modular kit,

FIG. 2 an exemplary guiding device of an exemplary modular kit,

FIG. 3 an exemplary guiding device of an exemplary modular kit,

FIG. 4 the exemplary guiding device of FIG. 2 from an alternative perspective,

FIG. 5 the exemplary guiding device of FIG. 2 from an alternative perspective,

FIG. 6 the exemplary guiding device of FIG. 3 from an alternative perspective,

FIG. 7 an exemplary guiding device of an exemplary modular kit,

FIG. 8 an exemplary guiding device of an exemplary modular kit,

FIG. 9 an exemplary guiding device of an exemplary modular kit,

FIG. 10A-10C exemplary second connecting elements,

FIG. 11 an exemplary computer device for designing a modular kit,

FIG. 12 an exemplary computer device for designing a modular kit, and

FIG. 13 an exemplary manufacturing system for manufacturing a modular kit.

### DETAILED DESCRIPTION

In the following, similar elements are identified with the same reference numerals.

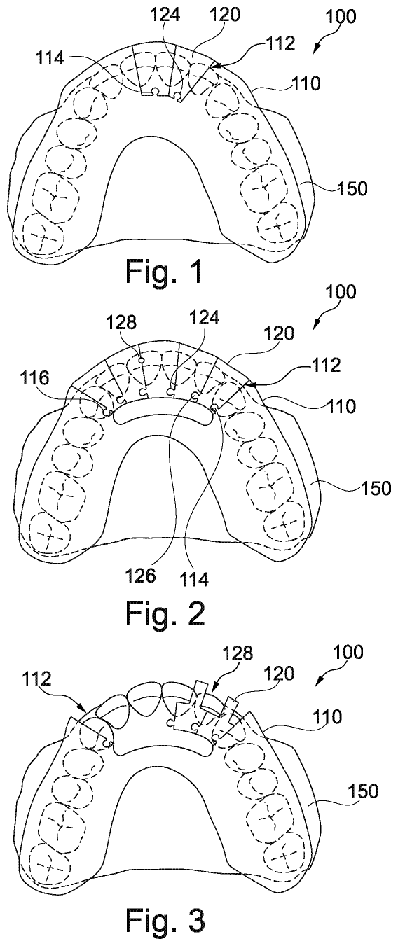

FIG. 1 shows an exemplary guiding device 100 of an exemplary modular kit. The guiding device 100 comprises a positioning splint 110 in the form of a patient-specific bite splint with a recess 112. The recess 112 is designed to expose an area of a patient's jaw 150 when the bite splint is placed on the patient's jaw 150. Guiding inserts 120 from a set of guiding inserts are arranged in the recess 112. When inserted, these guiding inserts 120 are positioned at predefined positions relative to the patient's jaw 150. Furthermore, the guiding inserts 120 are assigned to a processing step and, when inserted, are configured to provide guidance for one or more instruments during the corresponding processing step. By exchanging the guiding inserts 120 in the recess 112, the guiding device 100 may be converted into a guiding device 100 for another processing step. Thus, the positioning splint 110 of the modular kit allows a plurality of guiding devices 100 to be provided for a plurality of processing steps by exchanging the guiding inserts 120. The processing steps may be different processing steps of the same type and/or different processing steps.

In the example shown, the guiding inserts 120 are each attached to the positioning splint 110 by means of a non-destructive, detachable pin and socket connection. In the example shown, the positioning splint 110 comprises a connecting element 114 in the form of a projection or pin on an oral side of the recess 112 for each of the guiding inserts 120. The guiding inserts 120 each comprise a connecting element 124 in the form of a complementary receptacle or socket on an oral side. The connecting elements 114, 124 are assigned to each other in pairs. For example, the shapes of the connecting elements 114, 124 vary from pair to pair so that only connecting elements 114, 124 assigned to each other fit together.

FIG. 2 shows an exemplary guiding device 100 of an exemplary modular kit. The guiding device 100 comprises a positioning splint 110 in the form of a patient-specific bite splint with a recess 112. The recess 112 is designed to expose an area of a patient's jaw 150 when the bite splint is placed on the patient's jaw 150. Guiding inserts 120 from a set of guiding inserts are arranged in the recess 112. When inserted, these guiding inserts 120 are positioned at predefined positions relative to the patient's jaw 150. Furthermore, the guiding inserts 120 are assigned to a processing step and, when inserted, are configured to provide guidance for one or more instruments during the corresponding processing step. By exchanging the guiding inserts 120 in the recess 112, the guiding device 100 may be converted into a guiding device 100 for another processing step. Thus, the positioning splint 110 of the modular kit allows a plurality of guiding devices 100 to be provided for a plurality of processing steps by exchanging the guiding inserts 120. The processing steps may be different processing steps of the same type and/or different processing steps. In the example shown, each of the guiding inserts 120 is connected directly to the positioning splint 110 without the guiding inserts 120 being connected to each other. The guiding inserts 120 could also be connected to each other.

In the example shown, the guiding inserts 120 are each attached to the positioning splint 110 by means of a non-destructive, detachable pin and socket connection. In the example shown, the positioning splint 110 comprises a connecting element 114, 116 on opposite distal sides of the recess 112. In the example shown, one of the connecting elements 114 is designed in the form of a projection or pin. The other connecting element 116 is designed in the form of a receptacle or socket. The guiding inserts 120 each comprise a connecting element 124 in the form of a receptacle or socket and a connecting element 126 in the form of a projection or pin on opposite distal sides. The connecting elements 114, 124 and 116, 126 are assigned to each other in pairs. For example, the shapes of the connecting elements 114, 124 vary from pair to pair so that only connecting elements 114, 124 and 116, 126 assigned to each other fit together. In the example shown, the two outermost guiding inserts 120 of the guiding inserts 120 arranged next to each other in the receptacle 112 are each connected directly to the positioning splint 110. In addition, the guiding inserts 120 are each connected to the adjacent guiding insert(s) 120.

The guiding inserts 120 shown in FIG. 2 are guiding inserts 120 for inspecting preparation depths of one or more prepared teeth within the recess 112. For this purpose, the guiding inserts comprise recesses 128 for receiving one or more instruments for measuring the preparation depths.

FIG. 3 shows an exemplary guiding device 100 of an exemplary modular kit. The guiding device 100 comprises a positioning splint 110 in the form of a patient-specific bite splint with a recess 112. The recess 112 is designed to expose an area of a patient's jaw 150 when the bite splint is placed on the patient's jaw 150. Guiding inserts 120 from a set of guiding inserts are arranged in the recess 112. When inserted, these guiding inserts 120 are positioned at predefined positions relative to the patient's jaw 150. Furthermore, the guiding inserts 120 are assigned to a processing step and, when inserted, are configured to provide guidance for one or more instruments during the corresponding processing step. By exchanging the guiding inserts 120 in the recess 112, the guiding device 100 may be converted into a guiding device 100 for another processing step. Thus, the positioning splint 110 of the modular kit allows a plurality of guiding devices 100 to be provided for a plurality of processing steps by exchanging the guiding inserts 120. The processing steps may be different processing steps of the same type and/or different processing steps.

The guiding inserts 120 shown in FIG. 3 are guiding inserts 120 for identifying sections of several teeth to be prepared within the recess 112. The guiding inserts comprise recesses 128 whose contours define contours to be prepared of the sections to be prepared.

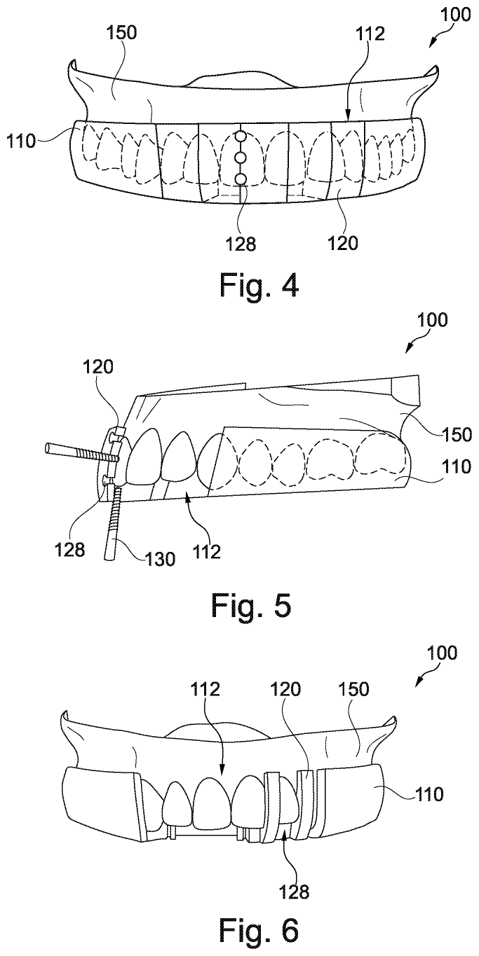

FIG. 4 shows the exemplary guiding device 100 of FIG. 2 from an alternative perspective. FIG. 5 shows the exemplary guiding device 100 of FIG. 2 from another alternative perspective. In the example in FIG. 5, not all guiding inserts 120 are inserted into the recess 112. In addition, the use of instruments 130 for measuring the preparation depths of the teeth within the recess 112 is illustrated. The local preparation depth of the teeth may be derived from how deep the instruments 130 may be inserted into the recesses 128.

FIG. 6 shows the exemplary guiding device 100 of FIG. 3 from an alternative perspective.

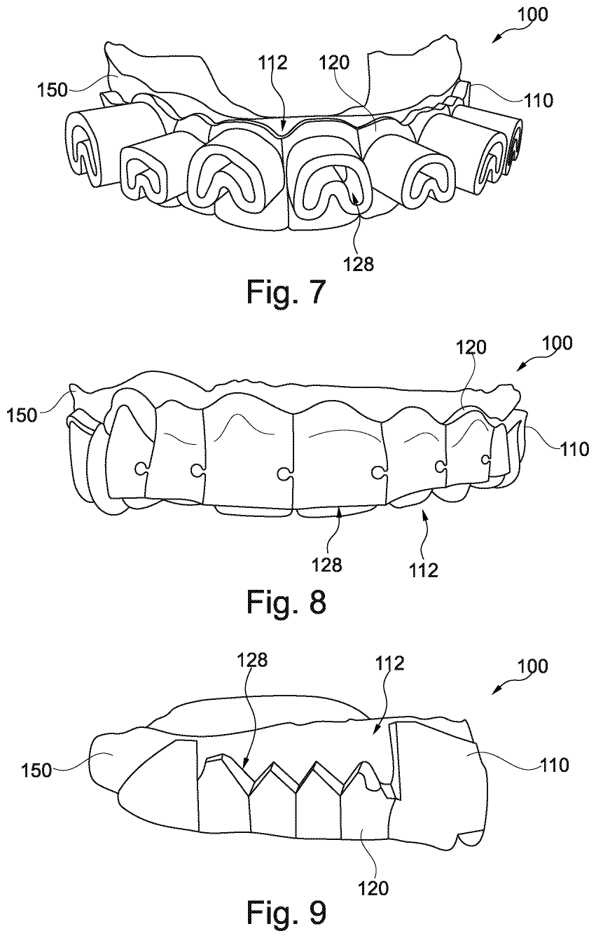

FIG. 7 shows an exemplary guiding device 100 of an exemplary modular kit. The guiding device 100 comprises a positioning splint 110 in the form of a patient-specific bite splint with a recess 112. The recess 112 is designed to expose an area of a patient's jaw 150 when the bite splint is placed on the patient's jaw 150. Guiding inserts 120 from a set of guiding inserts are arranged in the recess 112. When inserted, these guiding inserts 120 are positioned at predefined positions relative to the patient's jaw 150. Furthermore, the guiding inserts 120 are assigned to a processing step and, when inserted, are configured to provide guidance for one or more instruments during the corresponding processing step. By exchanging the guiding inserts 120 in the recess 112, the guiding device 100 may be converted into a guiding device 100 for another processing step. Thus, the positioning splint 110 of the modular kit allows a plurality of guiding devices 100 to be provided for a plurality of processing steps by exchanging the guiding inserts 120. The processing steps may be different processing steps of the same type and/or different processing steps.

The guiding inserts 120 shown in FIG. 7 are guiding inserts 120 for identifying sections to be prepared of several teeth within the recess 112. The guiding inserts comprise recesses 128 whose contours define contours to be prepared of the sections to be prepared.

FIG. 8 shows an exemplary guiding device 100 of an exemplary modular kit. The guiding device 100 comprises a positioning splint 110 in the form of a patient-specific bite splint with a recess 112. The recess 112 is designed to expose an area of a patient's jaw 150 when the bite splint is placed on the patient's jaw 150. Guiding inserts 120 from a set of guiding inserts are arranged in the recess 112. When inserted, these guiding inserts 120 are positioned at predefined positions relative to the patient's jaw 150. Furthermore, the guiding inserts 120 are assigned to a processing step and, when inserted, are configured to provide guidance for one or more instruments during the corresponding processing step. By exchanging the guiding inserts 120 in the recess 112, the guiding device 100 may be converted into a guiding device 100 for another processing step. Thus, the positioning splint 110 of the modular kit allows a plurality of guiding devices 100 to be provided for a plurality of processing steps by exchanging the guiding inserts 120. The processing steps may be different processing steps of the same type and/or different processing steps.

The guiding inserts 120 shown in FIG. 8 are guiding inserts 120 for identifying sections to be prepared of several teeth within the recess 112. The guiding inserts comprise recesses 128 whose contours define contours to be prepared of the sections to be prepared.

FIG. 9 shows an exemplary guiding device 100 of an exemplary modular kit. The guiding device 100 comprises a positioning splint 110 in the form of a patient-specific bite splint with a recess 112. The recess 112 is designed to expose an area of a patient's jaw 150 when the bite splint is placed on the patient's jaw 150. Guiding inserts 120 from a set of guiding inserts are arranged in the recess 112. When inserted, these guiding inserts 120 are positioned at predefined positions relative to the patient's jaw 150. Furthermore, the guiding inserts 120 are assigned to a processing step and, when inserted, are configured to provide guidance for one or more instruments during the corresponding processing step. By exchanging the guiding inserts 120 in the recess 112, the guiding device 100 may be converted into a guiding device 100 for another processing step. Thus, the positioning splint 110 of the modular kit allows a plurality of guiding devices 100 to be provided for a plurality of processing steps by exchanging the guiding inserts 120. The processing steps may be different processing steps of the same type and/or different processing steps.

The guiding inserts 120 shown in FIG. 9 are guiding inserts 120 for identifying one or more sections to be cut of the gingiva within the recess 112. The guiding inserts 120 comprise recesses 128 whose contours specify contours for cutting the sections to be cut of the gingiva. Furthermore, the recesses 128 may also specify angles for cutting the gingiva sections, for example. For this purpose, the guiding inserts 120 each have a chamfer at the edges of the recesses 128, the angle of which corresponds to the angle for cutting.

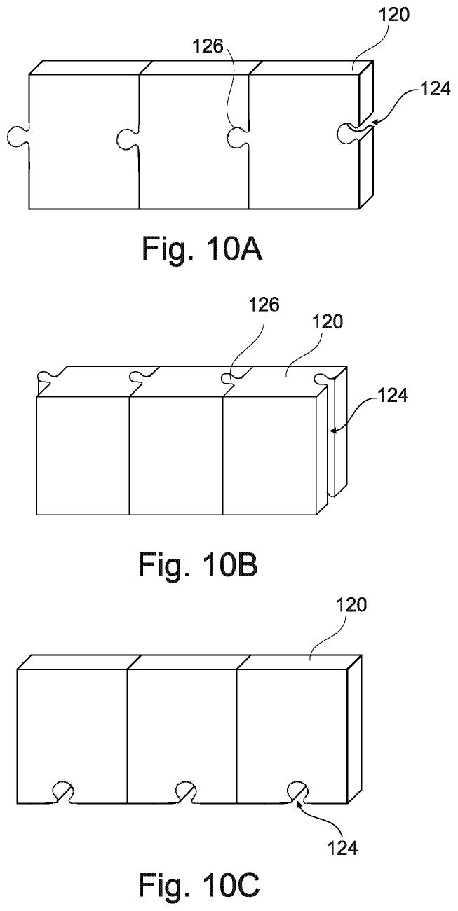

FIGS. 10A, 10B, and 10C show exemplary second connecting elements 124, 126 of guiding inserts 120. In FIG. 10A, the guiding inserts 120 each comprise, on opposite sides, for example on opposite distal sides, a connecting element 124 in the form of a receptacle or socket and a connecting element 126 in the form of a projection or pin. By means of these complementarily designed connecting elements 124, 126, the guiding inserts 120 may be connected to each other and, for example, via two outermost guiding inserts 120, to the positioning splint 110.

In FIG. 10B, the guiding inserts 120 comprise connecting elements 124, 126, which are designed as receptacles and projections. In the example shown, the receptacles 124 are each designed as grooves and the projections 126 are each designed as tongues. The connecting elements 124, 126 each have an elongated shape in a longitudinal direction. The pairs consisting of a projection and a receptacle designed to be complementary to the projection form, for example, pairs of complementary tongue and groove elements. This provides a type of rail system in which the components connected via the corresponding connecting elements 124, 126, in particular the guiding inserts 120, may be pushed into one another along a longitudinal direction of the groove and tongue or along a longitudinal direction of connecting element 126 and connecting element 124. This may have the advantage that a single guiding insert 120 may be easily and selectively pulled out again. Furthermore, guiding inserts 120 may also be connected to the positioning splint 110 via corresponding connecting elements 124, 126 designed as tongue and groove. In this case, the positioning splint 110 also comprises connecting elements designed as tongues and/or grooves. The longitudinal direction of the connecting elements 124, 126 and thus the insertion direction of the guiding inserts 120 may, for example, be a direction perpendicular to an occlusal surface and towards the patient's jaw. The longitudinal direction of the connecting elements 124, 126, and thus the insertion direction of the guiding inserts 120 may, for example, be an oral or vestibular direction.

In FIG. 10C, the guiding inserts 120 comprise a connecting element 124 in the form of a receptacle or socket on one side, for example on an oral side. These connecting elements allow the guiding inserts 120 to be connected, for example individually, to the positioning splint 110. Alternatively, the corresponding connecting elements could also be designed as projections or pins.

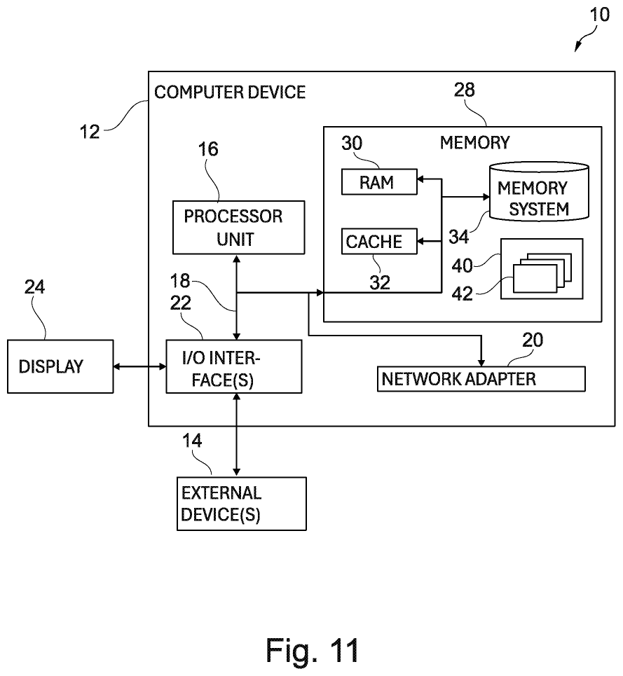

FIG. 11 shows a schematic representation of an exemplary computer device 10 for designing a modular kit for providing multiple guiding devices for multiple processing steps on oral tissue of a patient's jaw. The computer device 10 may be operated with numerous other general-purpose or special-purpose computer system environments or configurations.

The computer device 10 may be described in a general context of instructions executable by the computer device 10, such as program modules comprising executable program instructions executable by the computer device 10. The software may, for example, comprise routines, programs, objects, components, logic, data structures, etc., that perform specific tasks or implement specific abstract data types. The computer device 10 may be executed in distributed computing environments in which tasks are performed by remote processing devices connected to each other via a communication network. In a distributed computing environment, program modules may be located on both local and remote computer system storage media, including storage devices.

In FIG. 11, the computer device 10 is shown in the form of a universal computer device. The components of the computer device 10 may include, without limitation, one or more processor units or processors 16, a memory unit or system memory 28, and a bus 18 that couples various system components, including the memory unit 28, to the processor unit 16. The bus 18 represents one or more of various types of bus structures, including a memory bus or memory controller, a peripheral bus, an accelerated graphics port, and a processor or local bus utilizing any of a variety of bus architectures. By way of example and without limitation, such architectures include an Industry Standard Architecture bus (ISA bus), a Micro Channel Architecture bus (MCA bus), an Enhanced ISA bus (EISA bus), a local Video Electronics Standards Association bus (VESA bus), and a Peripheral Component Interconnect bus (PCI bus).

The computer device 10 may include a plurality of computer system-readable storage media or storage units. Such media may be any available storage media accessible by the computer device 10 and include both volatile and non-volatile storage media, removable and non-removable storage media.

A system memory 28 may include computer system-readable storage media in the form of volatile storage, such as random access memory (RAM) 30 and/or cache memory 32. The computer device 10 may further include other removable/non-removable, volatile/non-volatile computer system storage media. For example, a storage system 34 may be provided for reading from and writing to a non-removable, non-volatile magnetic medium, also referred to as a hard disk. For example, a magnetic disk drive may be provided for reading from and writing to a removable, non-volatile magnetic disk, such as a floppy disk, and an optical disk drive may be provided for reading from or writing to a removable, non-volatile optical disk, such as a CD-ROM, DVD-ROM, or other optical storage media. In such cases, each storage medium may be connected to the bus 18 via one or more disk interfaces. The system memory 28 may contain at least one program product with a set of program modules, for example, at least one program module designed to execute the designing of the modular kit.

The program 40 may comprise a set of one or more program modules 42 and may, for example, be stored in the memory 28. The program modules 42 may comprise an operating system, one or more application programs, other program modules, and/or program data. Each of the operating system, one or more application programs, other program modules and program data, or a combination thereof, may include an implementation of a network environment. One or more of the program modules 42 may perform the designing of the modular kit.

The designing of the modular kit comprises, for example, receiving a three-dimensional digital jaw model of the patient's jaw. The three-dimensional digital jaw model comprises, for example, the oral tissue of the patient's jaw to be processed in the course of the multiple processing steps. Three-dimensional digital processing models of the patient's jaw are generated, each of which defines a target state for the multiple processing steps on the oral tissue of the patient's jaw. A three-dimensional digital positioning splint model of the positioning splint of the kit is generated using the three-dimensional digital jaw model as a digital template for manufacturing the positioning splint of the kit. Furthermore, sets of at least one three-dimensional digital guiding insert model each for the sets of exchangeable patient-specific guiding inserts of the kit are generated as digital templates for manufacturing the sets of guiding inserts of the kit.

The computer device 10 may further communicate with one or more external devices 14, such as a keyboard, a pointing device such as a mouse, and a display 24, which enable a user to interact with the computer device 10. Such communication may be via input/output interfaces (I/O interfaces) 22. The computer device 10 may further communicate via the network adapter 20 with one or more networks, such as a local area network (LAN), a general wide area network (WAN), and/or a public network such as the Internet. The network adapter 20 may communicate with other components of the computer device 10 via the bus 18. It will be understood that, although not shown, other hardware and/or software components may be used in connection with the computer device 10.

The computer device 10 shown in FIG. 11 may be configured for designing the modular kit. The computer device 10 may be a standalone computer device without network connectivity that may receive data to be processed, such as scanned data of the patient's jaw and/or a three-dimensional digital jaw model, via a local interface. However, such an operation may also be performed using a computer device connected to a network, such as a communications network and/or a computing network.

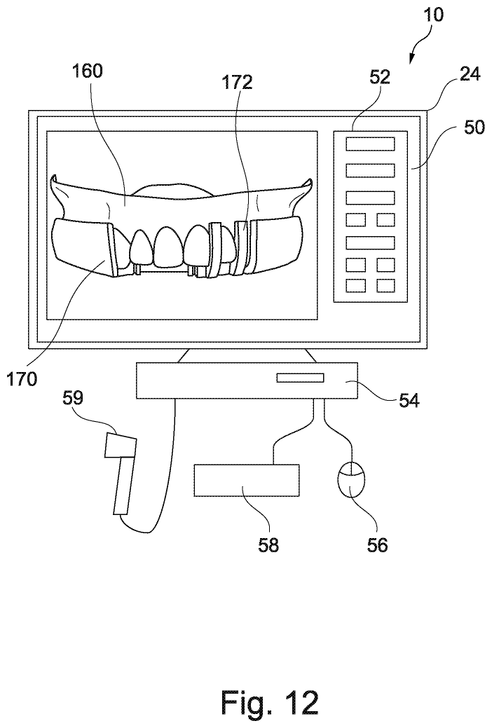

FIG. 12 shows an exemplary computer device 10 for designing a modular kit for providing multiple guidance devices for multiple processing steps on oral tissue of a patient's jaw. The computer device 10 may be configured, for example, as shown in FIG. 11. The computer device 10 may include a hardware component 54 comprising one or more processor units or processors and one or more memory units storing machine-executable program instructions. Execution of the program instructions by the one or more processor units may cause the one or more processor units to control the computer device 10 to generate three-dimensional digital processing models, a three-dimensional digital positioning splint model 170, and/or sets of at least one three-dimensional digital guiding insert model 172 each, respectively. For example, a three-dimensional digital jaw model 160 is used for this purpose. To provide the three-dimensional digital jaw model 160, scan data of the patient's jaw is used, for example, which is acquired using a scanning device such as an intraoral scanner. The computer device 10 may further include external devices such as a keyboard 58 and a mouse 56 that allow a user to interact with the computer device 10. Furthermore, the computer device 10 may include a display 24 that provides a user interface 50 with control elements 52 that allow the user to control the designing of the modular kit using the computer device 10. The structure of the computer device 10 may be shown on the user interface 50.

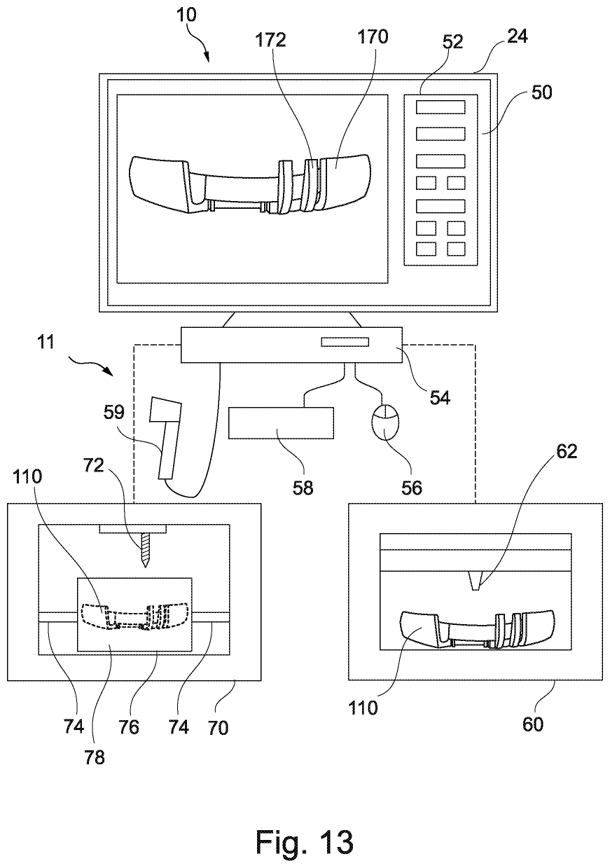

FIG. 13 shows a manufacturing system 11 for manufacturing a modular kit for providing multiple guiding devices for multiple processing steps on oral tissue of a patient's jaw. The manufacturing system 11 may comprise the computer device 10 of FIG. 12. The computer device 10 may further be configured to control one or more manufacturing devices for manufacturing the positioning splint of the kit and the sets of exchangeable patient-specific guiding inserts of the kit as physical copies of corresponding digital templates. The computer device 10 may further be configured to control a 3D printer 60 to manufacture one or more components of the modular kit according to the digital models 170, 172 designed on the computer device 10. The 3D printer 60 may include a printing element 62 that is designed to print the components of the modular kit, such as a positioning splint 110, layer by layer. For example, a plurality of 3D printers 60 may be used to print different components of the modular kit in parallel.

The computer device 10 may further be configured to control a machining device 70 for machining, which is configured to machine a blank 76 using one or more machining tools 72. The blank 76 made of raw material 78 may be provided using a holding device 74 and cut using the one or more machining tools 72 to perform a controlled material removal process into a desired final shape and size of the component to be manufactured, such as a positioning splint 110. For example, a plurality of machining devices 70 may be used to manufacture different components of the modular kit in parallel.

According to embodiments, the computer device 10 may be configured to control one or more 3D printers 60 in addition to one or more machining devices 70.

Although the invention has been illustrated and described in detail in the drawings and the foregoing description, such illustration and description are to be considered illustrative or exemplary and not limiting; the invention is not limited to the disclosed embodiments.

Further variations of the disclosed embodiments may be understood and realized by those skilled in the art in practicing the claimed invention, from a study of the drawings, the disclosure, and the appended claims. In the claims, the word “comprising” does not exclude other elements or steps, and the indefinite article “a” does not exclude a plurality. The mere fact that certain measures are listed in different dependent claims does not indicate that a combination of these measures cannot be advantageously applied.

A single processor or processor unit or other unit may perform the functions of several elements set forth in the claims. A computer program may be stored/distributed on a suitable medium, such as an optical storage medium or a solid-state storage medium, supplied together with or as part of other hardware, but may also be distributed in other forms, such as via the Internet or other wired or wireless telecommunications systems.

Those skilled in the art will understand that aspects of the present invention may be embodied as an apparatus or a computer program product. Accordingly, aspects of the present invention may take the form of a complete hardware embodiment, a complete software embodiment (including firmware, memory-resident software, microcode, etc.), or an embodiment combining software and hardware aspects, all of which may generally be referred to as a “circuit,” “module,” or “system.” Furthermore, aspects of the present invention may take the form of a computer program product embodied in one or more computer-readable media on which computer-readable code is embodied.

Any combination of one or more computer-readable media may be used. The computer-readable medium may be a computer-readable signal medium or a computer-readable storage medium. A “computer-readable storage medium” as used herein includes any tangible storage medium that may store instructions that may be executed by a processor or processor unit of a computer device. The computer-readable storage medium may be referred to as a computer-readable non-volatile storage medium. The computer-readable storage medium may also be referred to as a physical computer-readable medium. In some embodiments, a computer-readable storage medium may also be capable of storing data that may be accessed by the processor or processor unit of the computer device. Examples of computer-readable storage media include, but are not limited to: a floppy disk, a magnetic hard disk drive, a semiconductor hard disk, flash memory, a USB thumb drive, random access memory (RAM), read-only memory (ROM), an optical disk, a magneto-optical disk, and the processor's register file. Examples of optical discs include compact discs (CDs) and digital versatile discs (DVDs), such as CD-ROM, CD-RW, CD-R, DVD-ROM, DVD-RW, or DVD-R discs. Another example of an optical disc is a Blu-ray disc. The term “computer-readable storage medium” also refers to various types of recording media that may be accessed by the computer device via a network or communication link. Data may be accessed, for example, via a modem, the Internet, or a local area network. Computer-executable code embodied on a computer-readable medium may be transmitted using any suitable medium, including wirelessly, via a cable line, an optical fiber cable, RF, etc., or any suitable combination of the foregoing, but not limited thereto.

A computer-readable signal medium may comprise a propagated data signal embodying computer-executable code therein, for example in baseband or as part of a carrier wave. Such a propagated signal may take any of a variety of forms, including electromagnetic, optical, and any suitable combination thereof, but not limited thereto. A computer-readable signal medium may be any computer-readable medium that is not a computer-readable storage medium and that may communicate, propagate, or transport a program for use by or in connection with a system, apparatus, or device for executing instructions.

“Memory unit” or “memory” is an example of a computer-readable storage medium. A working memory is any memory that is directly accessible to a processor or processor unit. “Computer memory” is another example of a computer-readable storage medium. Computer memory is any non-volatile computer-readable storage medium. In some embodiments, computer memory may also be working memory and vice versa.

A “processor” or “processing unit” as used herein includes an electronic component that may execute a program or machine-executable instruction or computer-executable code. References to the computer device comprising a “processor” should be interpreted as possibly including more than one processor or processing core. For example, the processor may be a multi-core processor. A processor may also refer to a collection of processors within a single computer system or distributed across multiple computer systems. The term “computer device” should also be interpreted to potentially refer to a collection or network of computer devices, each comprising a processor or processors. The computer-executable code may be executed by multiple processors, which may be located within the same computer device or may even be distributed across multiple computer devices.

“Computer-executable code” may include machine-readable instructions or a program that causes a processor to perform an aspect of the present invention. Computer-executable code for performing operations for aspects of the present invention may be written in any combination of one or more programming languages, including an object-oriented programming language such as Java, Smalltalk, C++, or the like, and conventional procedural programming languages such as the programming language “C” or similar programming languages, and may be compiled into machine-executable instructions. In some cases, the computer-executable code may be in the form of a higher-level language or in a precompiled form and used together with an interpreter that spontaneously generates the machine-executable instructions.

The computer-executable code may be executed entirely on the user's computer device, partially on the user's computer device, as a separate software package, partially on the user's computer device and partially on a remote computer device, or entirely on the remote computer device or a server. In the latter scenario, the remote computer device may be connected to the user's computer device by any type of network, including a local area network (LAN) or a wide area network (WAN), or the connection may be made to an external computer device (for example, via the Internet using an Internet service provider).

In general, the program instructions may be executed on one processor or on multiple processors. In the case of multiple processors, these may be distributed across several different entities such as clients, servers, etc. Each processor could execute a portion of the instructions intended for that entity. Consequently, the terms “computer program” and “program instructions” when referring to a system or process comprising multiple entities should be understood to be adapted to be executed by a processor associated with or related to the corresponding entity.

A “user interface” as used herein is an interface that allows a user or operator to interact with a computer device. A “user interface” may also be referred to as a “human interface device.” A user interface may provide information or data to the operator and/or receive information or data from the operator. A user interface may enable an operator's inputs to be received by the computer device and may provide outputs from the computer device to the user. In other words, the user interface may enable an operator to control or operate a computer device, and the interface may enable the computer device to indicate the effects of the operator's control or operation. Displaying data or information on a display or graphical user interface is an example of providing information to an operator. Receiving data via a keyboard, mouse, trackball, touchpad, pointing stick, graphics tablet, joystick, gamepad, webcam, headset, gear stick, steering wheel, pedals, a wired glove, a dance mat, a remote control, one or more switches, one or more buttons, and an accelerometer are all examples of user interface components that enable the receipt of information or data from an operator.

A GUI element is a data object whose attributes specify the shape, layout, and/or behavior of an area displayed on a graphical user interface, such as a screen. A GUI element may be a standard GUI element such as a button, text box, tab, icon, text field, area, check box element or group of elements, or the like. A GUI element may equally be an image, an alphanumeric character, or any combination thereof. At least some of the properties of the displayed GUI elements depend on the data value that has been formed summarily for the group of data objects that the GUI element in question represents.

Aspects of the present invention are described with reference to illustrations and/or block diagrams of devices (systems) and computer program products according to embodiments of the invention. It is understood that each block or part of the blocks of the illustrations and/or block diagrams may be implemented by computer program instructions in the form of computer-executable code, if applicable. It is further understood that combinations of blocks in different illustrations and/or block diagrams may be combined, provided they are not mutually exclusive. These computer program instructions may be provided to a processor of a general-purpose computer, special-purpose computer, or other programmable data processing device, so that the commands executed via the processor or processor unit of the computer device or other programmable data processing device generate means for implementing the functions/actions specified in the flowchart and/or block diagram block or block diagram blocks.

These computer program instructions may also be stored in a computer-readable medium that may cause a computer device, another programmable data processing device, or other devices to function in a specific manner so that the instructions stored in the computer-readable medium produce an article for manufacture containing instructions that implement the function/action specified in the flowchart and/or block diagram block and/or block diagram blocks.

The computer program instructions may also be loaded onto a computer device, another programmable data processing device, or other devices to cause a series of operational steps to be performed on the computer device, the other programmable device, or other devices to produce a computer-implemented process, such that the instructions executed on the computer device or other programmable device provide processes for implementing the functions specified in the block diagram block or block diagram blocks.

### LIST OF REFERENCE SIGNS

10 computer device
11 manufacturing system
14 external device
16 processing unit
18 bus
20 network adapter
22 I/O interface
24 display
28 memory unit
30 RAM
32 cache
34 memory system
40 program
42 program module
50 user interface
52 control elements
54 hardware device
56 keyboard
58 mouse
60 3D printing device
62 printing element
70 machining device
72 machining tool
74 holding device
76 blank
78 raw material
80 scanning device
100 guiding device
110 positioning splint
112 recess
114 connecting element
116 connecting element
120 guiding inserts
124 connecting element
126 connecting element
128 recess
130 instrument
150 patient's jaw
160 3D jaw model
170 3D positioning splint model
172 3D guiding insert model

1. A modular kit for providing guiding devices for processing steps on oral tissue of a patient's jaw, wherein the kit comprises, as a common base for the guiding devices, a positioning splint in the form of a patient-specific bite splint with a recess which is designed to expose an area of the patient's jaw when the bite splint is placed on the patient's jaw,
wherein the kit further comprises sets of at least one exchangeable patient-specific guiding insert each, wherein the guiding inserts of the individual sets are positionable at predefined positions relative to the patient's jaw by insertion into the recess of the bite splint, wherein each of the sets of guiding inserts is each assigned to one of the processing steps, and the guiding insert of the corresponding set, when inserted, is configured to provide guidance for one or more instruments in the course of the processing step to which the corresponding set is assigned,
wherein the bite splint is reconfigurable by exchanging the one or more guiding inserts of one of the sets assigned to one of the processing steps, which are arranged in the recess, with the one or more guiding inserts of another one of the sets, which is assigned to another one of the processing steps, from a configuration as a guiding device for the one processing step into another guiding device for the other processing step.

2. The kit according to claim 1, wherein the bite splint comprises at least a first connecting element at an edge of the recess, wherein the guiding inserts of the sets each comprise at least a second connecting element at an edge, wherein the first and second connecting elements are configured to form non-destructively detachable first connections between at least a portion of the guiding inserts of the sets and the bite splint in order to hold the corresponding guiding inserts in position when the same are each arranged in the recess of the bite splint.

3. The kit according to claim 2, wherein at least some of the second connecting elements are further configured to form non-destructively detachable second connections between guiding inserts of the sets arranged in adjacent positions within the recess of the bite splint to hold the corresponding guiding inserts in the corresponding positions.

4. The kit according to claim 2, wherein the first connecting element is arranged in a region of the edge of the recess arranged on an oral side of the recess.

5. The kit according to claim 4, wherein at least a portion of the second connecting elements are each located in an area of the edges of the guiding inserts located on an oral side of the guiding inserts.

6. The kit according to claim 2, wherein the first connecting element is located in a region of the edge of the recess located on a vestibular side of the recess.

7. The kit according to claim 6, wherein at least a portion of the second connecting elements are each arranged in a region of the edges of the guiding inserts arranged on a vestibular side of the guiding inserts.

8. The kit according to claim 2, wherein at least a pair of first connecting elements is arranged in areas of the edge of the recess arranged on opposite distal sides of the recess.

9. The kit according to claim 8, wherein at least a portion of the second connecting elements are each arranged in pairs in areas of the edges of the guiding inserts arranged on opposite distal sides of the corresponding guiding inserts.

10. The kit according to claim 2, wherein the first and second connecting elements form pairs of mutually assigned connecting elements, the pairs each comprising a projection and a receptacle formed complementary to the projection, the pairs each being configured to establish one of the non-destructively detachable first or second connections.

11. The kit according to claim 1, wherein the recess of the bite splint is designed to expose a plurality of teeth and/or a portion of a gingiva and/or a portion of a jawbone of the patient's jaw.

12. The kit according to claim 1, wherein the sets of guiding inserts comprise guiding inserts of one or more of the following types for one or more processing steps of the following types:
a set with at least one guiding insert for identifying one or more sections of the jawbone within the recess to be prepared,
a set with at least one guiding insert for inspecting preparation depths of one or more prepared sections of the jawbone within the recess,
a set with at least one guiding insert for inspecting shapes of one or more prepared sections of the jawbone within the recess,
a set with at least one guiding insert for identifying one or more sections of one or more teeth within the recess to be prepared,
a set with at least one guiding insert for inspecting preparation depths of one or more prepared teeth within the recess,
a set with at least one guiding insert for inspecting shapes of one or more prepared teeth within the recess,
a set with at least one guiding insert for identifying one or more sections of the gingiva within the recess to be cut,
a set with at least one guiding insert for inspecting build-up thicknesses of one or more built-up teeth within the recess,
a set with at least one guiding insert for inspecting shapes of one or more cut sections of the gingiva within the recess,
a set with at least one guiding insert for identifying one or more sections to be built up on one or more teeth within the recess,
a set with at least one guiding insert for inspecting shapes of one or more built-up teeth within the recess,
a set with at least one guiding insert for applying trial restoration material for one or more trial restoration parts to one or more teeth within the recess,
a set with at least one guiding insert for inspecting trial restoration thicknesses of trial restoration material applied to one or more teeth within the recess,
a set with at least one guiding insert for inspecting shapes of trial restoration material applied to one or more teeth within the recess,
a set with at least one guiding insert for positioning one or more trial restoration parts on one or more teeth within the recess,
a set with at least one guiding insert for inspecting positions of one or more trial restoration parts on one or more teeth within the recess,
a set with at least one guiding insert for positioning one or more drills relative to the patient's jaw within the recess,
a set with at least one guiding insert for inspecting drilling results of drill holes inserted into the patient's jaw by one or more drills,
a set with at least one guiding insert for positioning one or more dental restoration parts on one or more prepared teeth within the recess,
a set with at least one guiding insert for inspecting positions of one or more restoration parts on one or more prepared teeth within the recess,
a set with at least one guiding insert for positioning one or more orthodontic appliances on one or more teeth within the recess,
a set with at least one guiding insert for inspecting positions of one or more orthodontic appliances on one or more teeth within the recess.

13. A computer device for designing a modular kit according to claim 1 for providing multiple guiding devices for multiple processing steps on oral tissue of a patient's jaw, wherein the computer device comprises a processor unit and a memory unit with stored machine-readable program instructions, wherein an execution of the program instructions by the processor unit causes the processor unit to control the computer device to
receive a three-dimensional digital jaw model of the patient's jaw,
generate three-dimensional digital processing models of the patient's jaw, each of which defines a target state for the multiple processing steps on the oral tissue of the patient's jaw,
generate a three-dimensional digital positioning splint model of the positioning splint of the kit using the three-dimensional digital jaw model as a digital template for manufacturing the positioning splint of the kit,
generate sets of at least one three-dimensional digital guiding insert model each for the sets of exchangeable patient-specific guiding inserts of the kit as digital templates for manufacturing the sets of guiding inserts of the kit.

14. The computer device according to claim 13, wherein an execution of the program instructions by the processor unit further causes the processor unit to control one or more manufacturing devices to manufacture the positioning splint of the kit and the sets of exchangeable patient-specific guiding inserts of the kit as physical copies of the corresponding digital templates.

15. A manufacturing system for manufacturing a modular kit according to claim 1 for providing a plurality of guiding devices for a plurality of processing steps on oral tissue of a patient's jaw, wherein the manufacturing system comprises a computer device and one or more manufacturing devices for manufacturing the positioning splint of the kit and the sets of exchangeable patient-specific guiding inserts of the kit, wherein the computer device comprises a processor unit and a memory unit with stored machine-readable program instructions, wherein an execution of the program instructions by the processor unit causes the processor unit to control the computer device to
receive a three-dimensional digital jaw model of the patient's jaw,
generate three-dimensional digital processing models of the patient's jaw, each of which defines a target state for the multiple processing steps on the oral tissue of the patient's jaw,
generate a three-dimensional digital positioning splint model of the positioning splint of the kit using the three-dimensional digital jaw model as a digital template for manufacturing the positioning splint of the kit, generate sets of at least one three-dimensional digital guiding insert model each for the sets of exchangeable patient-specific guiding inserts of the kit as digital templates for manufacturing the sets of guiding inserts of the kit, wherein an execution of the program instructions by the processor unit further causes the processor unit to control the one or more manufacturing devices to manufacture the positioning splint of the kit and the sets of exchangeable patient-specific guiding inserts of the kit as physical copies of the corresponding digital templates.

16. A computer program product for designing a modular kit according claim 1 for providing a plurality of guiding devices for a plurality of processing steps on oral tissue of a patient's jaw, wherein the computer program product comprises a computer-readable storage medium embodying machine-readable program instructions, wherein an execution of the program instructions by a processor unit of a computer device causes the processor unit to control the computer device to
receive a three-dimensional digital jaw model of the patient's jaw,
generate three-dimensional digital processing models of the patient's jaw, each of which defines a target state for the multiple processing steps on the oral tissue of the patient's jaw,
generate a three-dimensional digital positioning splint model of the positioning splint of the kit using the three-dimensional digital jaw model as a digital template for manufacturing the positioning splint of the kit,
generate sets of at least one three-dimensional digital guiding insert model each for the sets of exchangeable patient-specific guiding inserts of the kit as digital templates for manufacturing the sets of guiding inserts of the kit.

17. The computer program product according to claim 16, wherein an execution of the program instructions by the processor unit further causes the processor unit to control one or more manufacturing devices to manufacture the positioning splint of the kit and the sets of exchangeable patient-specific guiding inserts of the kit as physical copies of the corresponding digital templates.
